(19) United States
(12) Patent Application Publication
(10) Pub. No.: US 2009/0198198 A1
(43) Pub. Date: Aug. 6, 2009

(54) Method of treating skin for cellulite reduction

(76) Inventor: Susan Gardner, Somers Point, NJ (US)

Correspondence Address: HOLLSTEIN KEATING CATTELL JOHNSON & GOLDSTEIN P.C., WILLOW RIDGE EXECUTIVE OFFICE PARK, 750 ROUTE 73 SOUTH, SUITE 301, MARLTON, NJ, 08053, US

(21) Appl. No.: 12/012,425

(22) Filed: Feb. 4, 2008

(51) Int. Cl. A61M 35/00; A61K 33/14; A61P 17/00

(52) U.S. Cl. 604/290; 424/663

(57) ABSTRACT

A method is provided for the reduction of cellulite by means of loofah exfoliation. A rotatable, handheld loofah applicator device, used in combination with the unique application of a specific sequence of natural components, are applied daily, directly to targeted areas of the body where cellulite is located. This method removes cellulite formed on the skin, resulting in weight reduction and smoother, younger looking skin at the targeted areas of application.

### BACKGROUND OF THE INVENTION

The present invention relates to a method for the treatment of body tissue for reducing cellulite on targeted areas of the body.

Cellulite is a condition of the body most common to women, but found in men as well. It is located in many parts of the body, but is most prevalent on thighs, hips, stomach, buttocks, and upper arms. It is not caused by being overweight, as is commonly believed. Cellulite forms in the subcutaneous level of skin tissue. Fat cells are arranged in chambers surrounded by bands of connective tissue. As water is retained, fat cells held within the perimeters of this area expand and stretch the connective tissue. Eventually this connective tissue contracts and hardens, holding the skin at a non-flexible length, while the surrounding tissue continues to expand with weight, or water gain. This results in areas of the skin being held down while other sections protrude outward, resulting in bulges and lumpy areas on the body. Cellulite presents a very unsightly appearance. As a result, there is a strong demand for cellulite reducing treatments.

There have been many prior means developed to accomplish cellulite reduction, including radiation emitting techniques, electrostatic applications, various types of message and heat therapies, and even surgery. While some of these have had a degree of success, the limited success which has been attained has come with a number of serious drawbacks in the form of health risks, extreme discomfort and pain, and high monetary expense.

The use of loofahs have also been directed to the removal of dead skin. However, most loofahs are handheld and used manually, which results in uneven application. In addition, proper usage of manual loofahs is time consuming and so they are usually underused or, in most cases, misused. As a result, they are generally ineffective for meaningful cellulite removal. Most importantly, there is no method for using any type of loofah with cellulite reducing components, in an orderly, effective fashion, in order to ensure a steady, consistent reduction in cellulite from the surface of the body.

### SUMMARY OF THE INVENTION

It is thus the object of the present invention to address the unsightly and unhealthy buildup of cellulite in the body and to overcome the limitations and disadvantages of prior cellulite reduction techniques and methods.

It is an object of the present invention to provide a cellulite reduction method which is safe to use and highly effective in the reduction of body cellulite.

It is another object of the present invention to provide a cellulite reduction method which is non-invasive, avoids the use of harmful chemicals or treatments, but is, in fact pleasant on the body.

It is still another object of the present invention to provide a cellulite reduction method which can be readily and easily performed by women and men on a regular basis, everyday.

It is a further object of the present invention to provide a cellulite reduction method which employs a handheld device, inexpensive to obtain and easy to maintain, which employs a loofah which is comfortably rotatable on the body.

It is another object of the present invention to provide a cellulite reduction method which employs natural components, inexpensive and readily replaceable, which are applied directly to the body and on which the handheld loofah device interacts.

It is another object of the present invention to provide a cellulite reduction method which not only quickly reduces cellulite on the body, but, upon continued use and application, keeps cellulite from reforming.

These and other object are accomplished by the present invention, a method for the reduction of cellulite by means of loofah exfoliation. A rotatable, handheld loofah device, used in combination with the unique application of a specific sequence of natural components, are applied daily, directly to targeted areas of the body where cellulite is located. This method removes cellulite formed on the skin, resulting in weight reduction and smoother, younger looking skin at the areas of application.

The novel features which are considered as characteristic of the invention are set forth in particular in the appended claims. The invention, itself, however, both as to its design, construction and use, together with additional features and advantages thereof, are best understood upon review of the following detailed description with reference to the accompanying drawings.

### BRIEF DESCRIPTION OF THE DRAWINGS

FIG. 1 is an elevation of the loofah applicator device used in the method of the present invention.

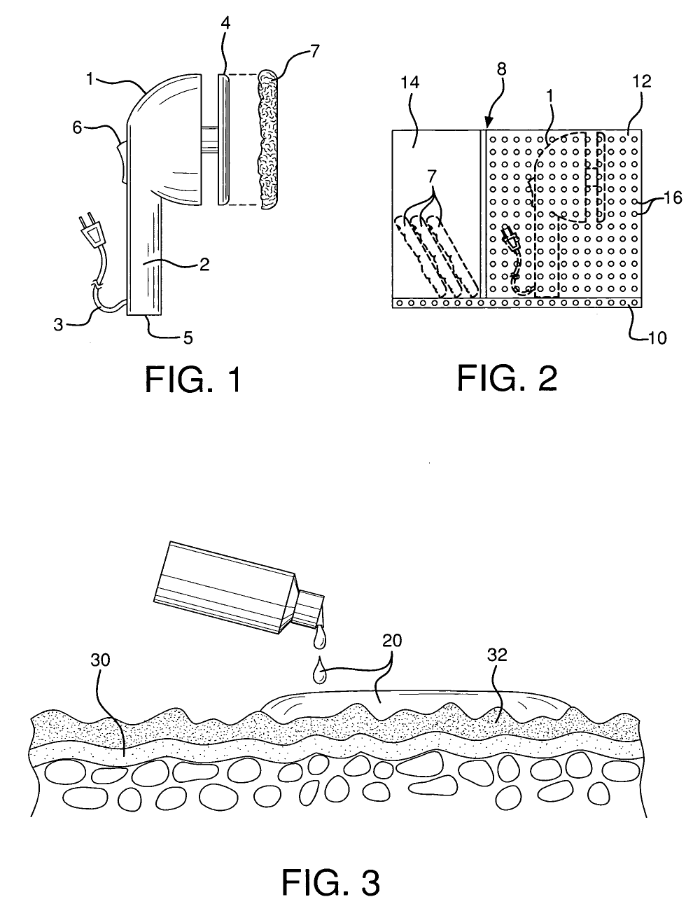

FIG. 2 is an elevation of the loofah applicator device and holder used in the method of the present invention.

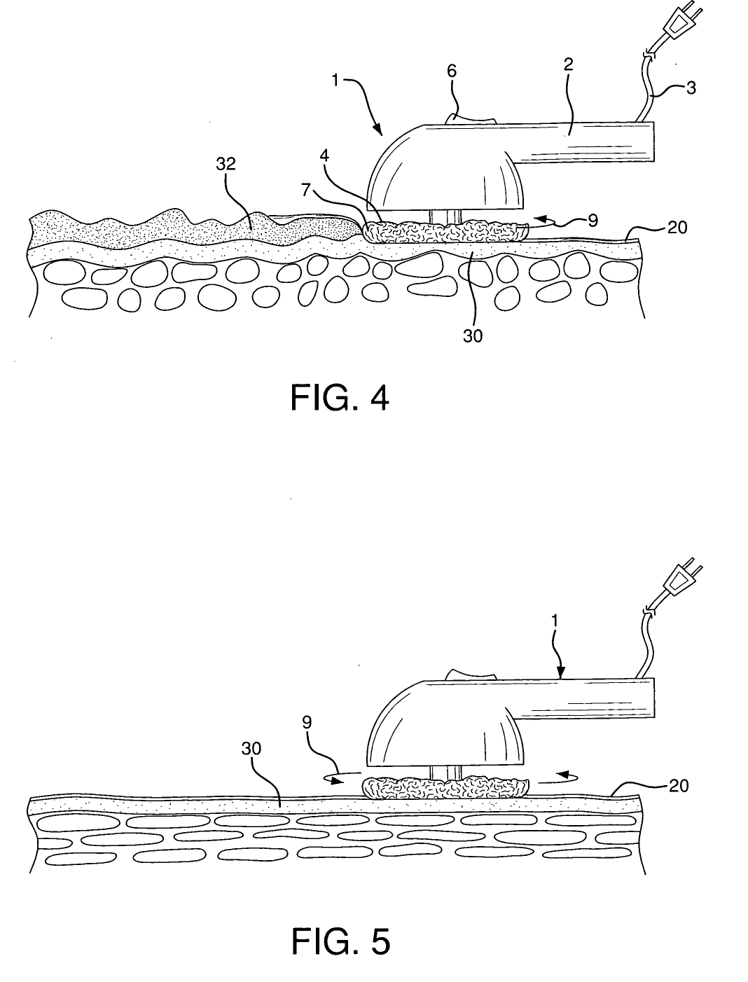

FIGS. 3-7 depict the steps in the method of the present invention.

### DETAILED DESCRIPTION OF THE INVENTION

Handheld electrically rechargeable loofah applicator device 1, integral to the method of the present invention, comprises body section 2 in which a battery powered drive motor and drive assembly (not shown) are housed. Electrical charging cord 3 extends from body section 2, for recharging an internal battery located within the body section. The drive motor and drive assembly serve to rotate and spin head 4 of loofah device 1 at low and high speed. The battery, motor and drive assembly system necessary for the spinning and rotation of head 4 is well known, the electrical and gearing systems in and of themselves, not forming a part of the invention. For instance, the drive assembly may comprise a drive shaft with necessary gearing, operatively connected to the drive motor, and to head 4, to ensure for appropriate head rotation. However, significant to the invention is that head 4 is operational for high speed rotatable motion, resulting in comfortable, yet firm application on the body of the user.

While loofah device 1 is not to be considered restricted to any one configuration, it is contemplated that a representative device would be lightweight and configured to be easily held with one hand. Body section 2 would be waterproof and have a non-slip outer surface made of soft rubber, plastic, or the like. Body section 2 has a flat bottom surface 5, so as to allow it to be stood upright. On/off/high/low switch 6, to rotatably actuate head 4 and cease its rotation, would be readily accessible.

Head 4 of applicator device 1 is configured to snugly accept replaceable loofah pad 7. Designed to tightly fit around head 4 while in use, loofah pad 7 is also readily removeable when worn and replaceable with a new, fresh loofah pad.

Applicator device 1 can be stored in holder 8 which, in the preferred configuration, has a suction bottom 10, for removeable attachment to a shelf or side of a bathtub, and two compartments. Compartment 12 is configured to house applicator device 1 and compartment 14 stores loofah pads 7. Perforated holes 16 provide aeration for applicator device 1.

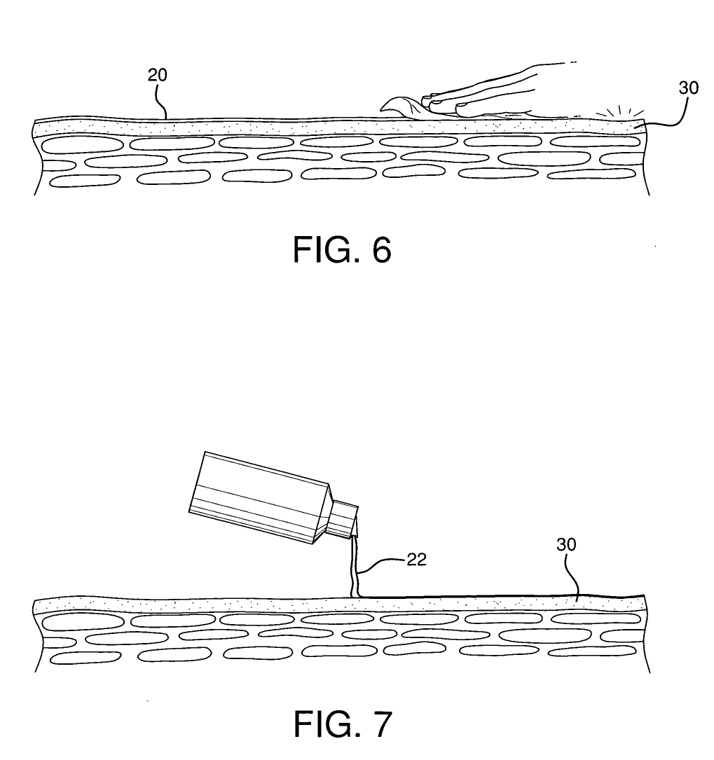

The method of the present invention first begins, as seen in FIG. 3, with the application of an exfoliating scrub solution 20, a natural wash such as a mixture of sea salt and an exfoliator, on targeted skin area 30 where cellulite 32 is present. This mixture is worked into a lather on the skin area 30, which starts the process of removing cellulite 32.

As shown in FIG. 4, applicator device 1, with loofah pad 7 mounted on head 4, is then actuated by setting switch 6, to commence rotation and spinning 9 of the head. The user, holding body 2 of applicator device 1, manually moves head 4 with loofah pad 7 thereon, in a circular motion directly on targeted skin area 30, effectively continuing the process of removal of cellulite 32. The lightweight nature of handheld applicator device 1 allows the user to continue the circular motion of spinning and rotating head 4 with loofah pad 7 thereon, on target skin area 30 for two to three minutes or a longer period, if desired, with minimal physical labor; thereby increasing the effectiveness of the process and ultimately eliminating cellulite from skin area 30, as depicted in FIG. 5.

Exfoliating scrub solution 20 is then removed and target skin area 30 is patted dry. See FIG. 6. Skin moisturizer 22 is applied on target skin area 30, to moisturize and seal the treated skin area, protecting it from further aging and damage. It is contemplated that skin moisturizer 22 will be a cooling, intensive firming cream.

The herein described method is done at least once a day and, optimally, twice a day, for more rapid results. It can advantageously be performed either in or out of a bath or shower. Loofah pads 7 should be replaced often in order to maintain a consistent, constant rough surface for most effective and efficient cellulite elimination.

Thus, the method of the present invention, used in conjunction with loofah applicator device 1, serves to seriously reduce cellulite and the unsightly skin which accompanies it. It gives treated skin a healthy, younger looking appearance, with a minimum of effort. The lightweight, ergonomic configuration of applicator device 1 itself, allows for even, consistent rotations, enabling the user to target areas more prone to cellulite. Since applicator device 1 is much easier to handle than a manual loofah, it is more efficiently used, and hence more effective. It allows the user to perform the method for an extended period of time and more often. The areas of cellulite are thus targeted longer and can provide more dramatically successful results.

Certain novel features and components of this invention are disclosed in detail in order to make the invention clear in at least one form thereof. However, it is to be clearly understood that the invention as disclosed is not necessarily limited to the exact form and details as disclosed, since it is apparent that various modifications and changes may be made without departing from the spirit of the invention.

1. A method of treating the skin of an individual for cellulite reduction, the method comprising the steps of:
providing an exfoliating scrub solution;
applying the exfoliating scrub solution to a targeted area of the skin;
removing cellulite by the application of the exfoliating scrub solution;
providing a rotatable hand-held appliance with a high speed rotatable loofah element;
rotating the loofah element at high speed directly on the targeted area of the skin on which the exfoliating scrub solution was applied;
reducing cellulite in the targeted skin area by means of the high speed rotation of the loofah element on the target area of skin on which the exfoliating scrub solution was applied;
removing the exfoliating scrub solution from the target area of the skin;
providing a skin moisturizer;
applying the skin moisturizer to the targeted area of the skin; and
sealing and protecting the targeted area of the skin by the application of the skin moisturizer.

2. The method as in claim 1 further repeating the steps of claim 1 on multiple target areas of the skin prone to the existence of cellulite.

3. The method as in claim 1 further repeating the steps of claim 1 two times each day.

4. The method of claim 1 wherein the loofah element is rotated at high speed on the targeted area of the skin for a period of two to three minutes.

5. The method as in claim 1 wherein the exfoliating scrub solution comprises a sea salt lather and exfoliator.

6. The method as in claim 1 wherein the skin moisturizer comprises a cooling, intensive firming cream.

7. The method as in claim 1 comprising the additional steps of removing the loofah element from the appliance after several uses and providing a replacement loofah element for use on the appliance.
